(19) United States
(12) Patent Application Publication
(10) Pub. No.: US 2026/0315478 A1
(43) Pub. Date: Oct. 15, 2026

(54) Composition Combination For Use In The Treatment And/Or Prevention Of Hair Loss

(71) Applicant: LEGACY HEALTHCARE (SWITZERLAND) SA, Epalinges (CH)

(72) Inventor: Saad HARTI, Lutry (CH)

(21) Appl. No.: 19/484,053

(22) PCT Filed: May 16, 2024
(86) PCT No.: PCT/EP2024/063618

§ 371 Date: Nov. 13, 2025

Related U.S. Application Data
(60) Provisional application No. 63/467,076, filed on May 17, 2023.

(30) Foreign Application Priority Data
May 17, 2023 (EP) 23174049.9

(51) Int. Cl. A61K 31/519; A61K 36/185; A61K 36/752; A61K 36/77; A61K 36/8962; A61P 17/14

(52) U.S. Cl. CPC A61K 31/519; A61K 36/5777; A61K 36/752; A61K 36/77; A61K 36/8962; A61P 17/14; A61K 2236/333

(57) ABSTRACT

The invention relates to a composition combination for use in the treatment and/or prevention of hair loss in a subject. Also disclosed are kits and methods of treating and/or preventing hair loss using said composition combination.

### FIELD OF THE INVENTION

The invention relates to a composition combination for use in the treatment and/or prevention of hair loss in a subject. Also disclosed are kits and methods of treating and/or preventing hair loss using said composition combination.

### BACKGROUND OF THE INVENTION

JAK (Janus kinase) inhibitors are a class of drugs whose mechanism of action consists of suppressing intracellular signaling mediated by multiple cytokines. These cytokines operate through the JAK/STAT (signal transducer and activator of transcription) pathway that plays critical roles in orchestrating immune system. Dysregulation of the JAK-STAT pathway may result in pathological processes of various immune and inflammatory disorders. Oral and topical JAK inhibitors are approved for the treatment of various autoimmune and inflammatory mediated chronic conditions, including alopecia areata (AA), an immune mediated disease characterized by patchy or total loss of scalp and/or body hair.

The chronic use of JAK inhibitors has been documented to trigger serious side effects (Hoisnard, 2022), including serious infections, major adverse cardiovascular event (MACE), thrombosis, malignancy and mortality, as mentioned in the prescribing information of several JAK inhibitors, including baricitinib (Olumiant®), and ritlecitinib (Litfulo®), two JAK inhibitor approved for the treatment of AA (prescribing information).

Based on observed long term health risks associated with the chronic use of JAK inhibitors, the US Food and Drug administration issued a warning in 2021 and imposed a black box warning to all drugs belonging to the JAK inhibitors class (FDA warning). The European Medicines Agency followed in 2022 (EMA warning).

Since the long-term exposure to JAK inhibitors is associated with serious health risk, a rational medical approach consists of discontinuing the risk-prone treatment once the patient's symptoms have been cleared. In the specific case of AA, this would consist of discontinuing JAK inhibitor treatment once the patient's hair have grown back. However, data have shown that once that treatment with JAK inhibitors is discontinued, hair falls back again within less than three months after treatment discontinuation (Yan 2022).

There is high unmet medical need for a treatment which would allow AA patients who respond to JAK inhibitors to discontinue the treatment upon clearance of symptom, therefore reducing the long term associated health risks, without suffering from a recurrence of hair loss.

### SUMMARY OF THE INVENTION

This object has been achieved by providing a composition combination for use in the treatment and/or prevention of hair loss, comprising,
i) a first pharmaceutical composition comprising a therapeutically effective amount of at least one Janus kinase (JAK) inhibitor and,
ii) a second pharmaceutical composition comprising, as active ingredients, therapeutically effective amounts of an extract of Allium species, an extract of Citrus species, an extract of Paullinia species and an extract of Theobroma species,
wherein the first and second pharmaceutical compositions are administered concomitantly, separately or staggered in time to a subject in need thereof.

A further object of the present invention is to provide a method of treatment and/or prevention of hair loss in a subject comprising, administering i) a first pharmaceutical composition comprising a therapeutically effective amount of at least one Janus kinase (JAK) inhibitor and, ii) a second pharmaceutical composition comprising, as active ingredients, therapeutically effective amounts of an extract of Allium species, an extract of Citrus species, an extract of Paullinia species and an extract of Theobroma species, wherein the first and second pharmaceutical compositions are administered concomitantly, separately or staggered in time to a subject in need thereof.

A further object of the present invention is to provide the use of a composition of the invention in the manufacture of a medicament for the treatment and/or prevention of hair loss in a subject, comprising, administering i) a first pharmaceutical composition comprising a therapeutically effective amount of at least one Janus kinase (JAK) inhibitor and, ii) a second pharmaceutical composition comprising, as active ingredients, therapeutically effective amounts of an extract of Allium species, an extract of Citrus species, an extract of Paullinia species and an extract of Theobroma species, wherein the first and second pharmaceutical compositions are administered concomitantly, separately or staggered in time to a subject in need thereof.

A further object of the present invention is to provide a kit for the treatment and/or prevention of hair loss comprising a combination composition, or combination composition for use, of the invention.

### DESCRIPTION OF THE FIGURES

FIG. 1 SALT scoring example (adapted from Olsen, 2004)

FIG. 2 SALT score of the composition-treated patients continued to improve during and after treatment, and the change in SALT score was continuous and statistically significantly superior to placebo after 24 weeks of treatment and throughout the treatment-free follow-up period.

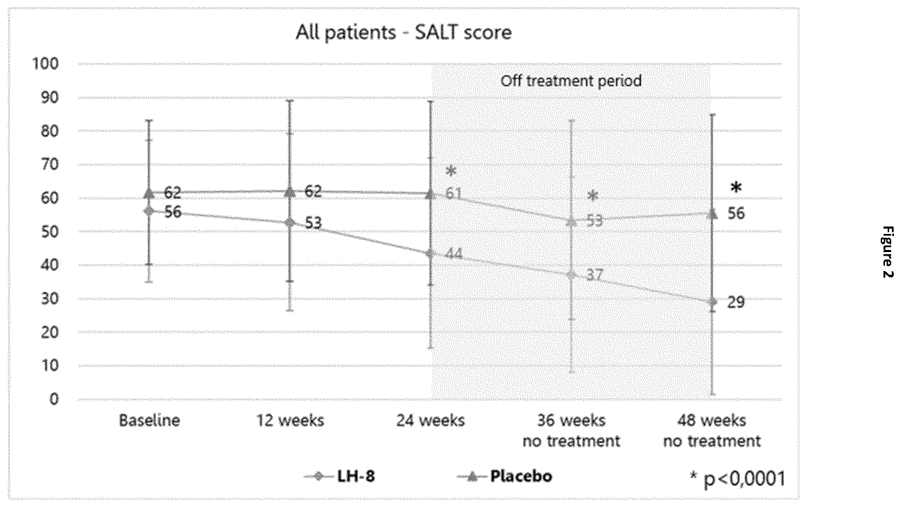

### DESCRIPTION OF THE INVENTION

Although methods and materials similar or equivalent to those described herein can be used in the practice or testing of the present invention, suitable methods and materials are described below. All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control. Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “comprise/comprising” is generally used in the sense of include/including, that is to say permitting the presence of one or more features or components. The terms “comprise(s)” and “comprising” also encompass the more restricted ones “consist(s)”, “consisting” as well as “consist/consisting essentially of”, respectively.

As used in the specification and claims, the singular form “a”, “an” and “the” include plural references unless the context clearly dictates otherwise.

As used herein the terms “subject”/“subject in need thereof”, or “patient”/“patient in need thereof” are well-recognized in the art, and, are used interchangeably herein to refer to a mammal, including dog, cat, rat, mouse, monkey, cow, horse, goat, sheep, pig, camel, and, most preferably, a human. In some cases, the subject is a subject in need of treatment or a subject with a disease or disorder. However, in other aspects, the subject can be a normal subject. The term does not denote a particular age or sex. Thus, adult and newborn subjects, whether male or female, are intended to be covered. Preferably, the subject is a human, most preferably a human suffering from hair loss or a human that might be at risk of suffering from hair loss.

The term “about,” particularly in reference to a given quantity, number or percentage, is meant to encompass deviations of plus or minus twenty percent (±20%), preferably plus or minus ten percent (±10%). For example, about 5 encompasses any value between 4.5 to 5.5, such as 4.5, 4.6, 4.7, 4.8, 4.9, 5, 4.1, 5.2, 5.3, 5.4, or 5.5.

As used herein, “at least one” means “one or more”, “two or more”, “three or more”, etc. For example, at least 8 weeks means 8 weeks or more i.e., 9 weeks, 10 weeks, 11 weeks, etc.

In one aspect, the invention provides a composition combination for use in the treatment and/or prevention of hair loss, comprising,
i) a first pharmaceutical composition comprising a therapeutically effective amount of at least one Janus kinase (JAK) inhibitor and,
ii) a second pharmaceutical composition comprising, as active ingredients, therapeutically effective amounts of an extract of Allium species, an extract of Citrus species, an extract of Paullinia species and an extract of Theobroma species,
wherein the first and second pharmaceutical compositions are administered concomitantly, separately or staggered in time to a subject in need thereof.

In one aspect, the first and the second pharmaceutical compositions are administered concomitantly, i.e. co-administered, at the same time. For example, the first and the second pharmaceutical compositions are administered from the beginning and as long as necessary or only during a period of time. In this case, administration of the first pharmaceutical composition is discontinued once one or more therapeutic effects are detected, usually after 24 to 36 weeks, whereas the second composition continues to be administered.

In another example, the first pharmaceutical composition is usually administered first, and the second pharmaceutical composition is administered thereafter, usually within weeks (e.g. about 1, about 2, about 3, about 4, about 5, about 10, about 20, about 30, weeks). In this case, administration of the first pharmaceutical composition is discontinued once one or more therapeutic effects are detected, e.g. usually after 24 to 36 weeks, whereas the second composition continues to be administered.

In another aspect, the first and the second pharmaceutical compositions are administered staggered in time, e.g. there is no overlap in the administration as the second pharmaceutical composition is administered after the last administration of the first pharmaceutical composition. Usually, the second pharmaceutical composition is administered about one hour, about 1 day, about 4 days, about one week, about 2 weeks, about 3 weeks, about 5 weeks, about 10 weeks, about 15 weeks, about 20 weeks or more, after the last administration of the first pharmaceutical composition.

In one aspect, the first pharmaceutical composition is administered for a period of time necessary to detect one or more therapeutic effects consisting of slowing down the loss of hair, stimulating its growth and/or increasing the density of hair, as evidenced by measuring the severity of alopecia tool (SALT) score.

Usually, said period of time is comprised between about 16 to about 48 weeks, preferably between about 20 to about 40 weeks, more preferably between about 20 and about 36 weeks and even more preferably about 36 weeks.

Any method known in the art may be used to detect, monitor and/or evidence the one or more therapeutic effects consisting of slowing down the loss of hair, stimulating its growth and/or increasing the density of hair.

In one aspect, the one or more therapeutic effects are detected, monitored and/or evidenced by measuring the severity of alopecia tool (SALT) score.

In one aspect, the SALT score is determined or measured before of the starting the administration of the first pharmaceutical composition or of the combination composition.

In one aspect, the SALT score is determined or measured at the beginning of the starting the administration of the first pharmaceutical composition or of the combination composition. SALT scoring is a calculation based on a scoring system. Scalp is divided into four areas: left side of scalp representing 18% of the scalp surface area, right side of scalp—18% of scalp surface area; top—40% of scalp surface area and back—24% of scalp surface area. The percentage of hair loss in each of the four scalp areas is determined independently, each multiplied by its coefficient (left and right side: 0.18 each; top: 0.40 and back 0.24). The coefficient of each area varies according to the location. Then the resulting hair loss percentages of all four areas are summed up for a final total % hair loss, designated as the SALT score.

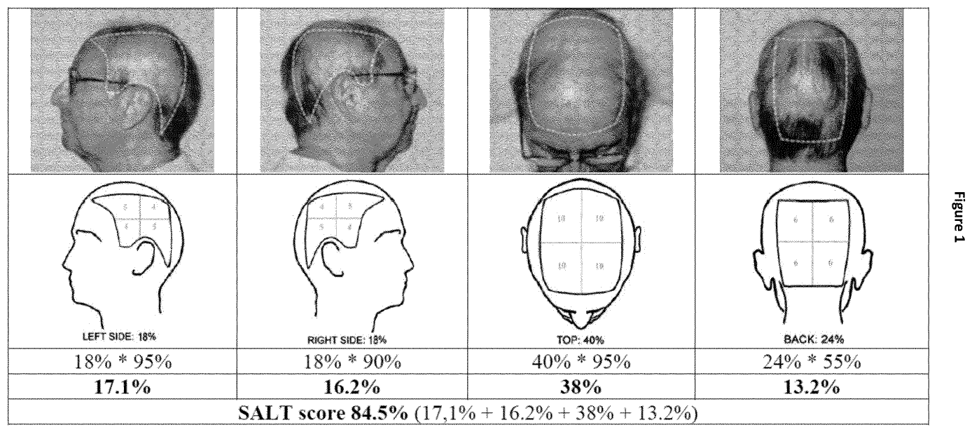

Olsen et al. also described a SALT score assessment more suitable for clinical trials. Each area is further subdivided into four quadrants (5%+4%+4%+5%; 10%+10%+10%+10% or 6%+6%+6%+6%) according to the scalp area, see FIG. 1.

The percentage of hair loss in each quadrant is determined independently, each multiplied by its coefficient (0.04, 0.05, 0.06 or 0.1). The coefficient of each quadrant varies according to the location (FIG. 1). Then the resulting hair loss percentages of all four areas (16 quadrants) are summed up for a final total percentage of hair loss, designated as the SALT score.

Due to the numerous calculations to obtain final SALT score and due to the variation of the coefficient depending on the area, manual SALT scoring might be at risk of errors. In one aspect, the SALT scoring is thus computer implemented. In a preferred aspect, the SALT score assessment described by Olsen is used to detect, monitor and/or evidence the one or more therapeutically effects.

Usually, a subject's baseline SALT score is determined before starting the administration of a composition the invention.

In one aspect, a decrease of at least about 2% or more, at least about 5% or more, at least about 10% or more, at least about 15% or more, at least about 20% or more, at least about 30% or more, at least about 40% or more, or at least about 50% or more in SALT score is detected when compared to the subject's baseline SALT score determined before starting the administration of the first pharmaceutical composition.

Usually, the administration of the first composition is discontinued after 24 to 36 weeks, preferably after 28 weeks, more preferably after 32 weeks, even more preferably after 36 weeks. In one aspect, the second composition continues to be administered. In another aspect, the administration of the second composition is started.

In one aspect, the first and second compositions are co-administered (i.e. concomitantly) for at least 1 hour, at least 1 day, at least 4 days, at least one week, at least 2 weeks, at least 3 weeks, at least 5 weeks, at least 10 weeks, at least 15 weeks, at least 20 weeks, at least 25 weeks, at least 36 weeks or more.

The term “treatment” or “treating” means any administration of one or more compositions, pharmaceutical compositions, therapeutic agents, active ingredients, compounds, etc. . . . of the disclosure to a subject for the purpose of:
(i) inhibiting the disease, that is, arresting the development of clinical symptoms;
(ii) reversing the disease, and/or
(iii) relieving the disease, that is, causing the regression of clinical symptoms.

As used herein, the term “prevention” or “preventing” means any administration of one ore more compositions, pharmaceutical compositions, therapeutic agents, active ingredients, compounds, etc. . . . of the disclosure to a subject for the purpose of preventing the disease, that is, causing the clinical symptoms and signs of the disease not to develop.

In the context of the present invention, the disease is hair loss. In an aspect, the hair loss is an immune-mediated disease, preferably an autoimmune disease. More preferably, the autoimmune disease is Alopecia Aerata.

Alopecia Areata (AA) is an immune-mediated, auto-immune, inflammatory hair disease affecting both paediatric and adult patients. The immunoinflammatory attack of scalp hair follicles aborts normal hair cycling leading to their early entry in catagen and telogen and hair loss. The disease starts with the occurrence of a perifollicular inflammation and invasion of immunoinflammatory cells around the anagen hair bulbs causing HF immune privilege collapse, tissue dystrophy, HF cell death, and hair shaft shedding (Lintzeri-2022). A central role is played by NKG2D+T cells and natural killer (NK) cells and autoreactive CD8+T-lymphocytes that recognize autoantigens upon exposure of MHC I, II expression on follicular cells. T-cell activation is accompanied by an elevated IFN-γ secretion recruiting or activating more inflammatory cells including macrophages, mast cells, and dendritic cells leading to accelerated cell death and apoptosis of hair follicular cells (Bertolini, M., 2020).

The early clinical manifestations of AA appear as random, patchy hair loss—that may result sometimes in total scalp hair loss (alopecia totalis) or even become generalized to all body parts (alopecia universalis). AA affects patients from all ethnicities, but more women than men, with similar clinical manifestations. Most importantly, when AA starts early in infancy, its prognosis is usually more severe and associated with a higher frequency of unpredictable relapses during adulthood (Villasante Fricke, A. C., 2015).

In one aspect, the subject in need thereof is a subject suffering from Alopecia Totalis/Universalis (SALT score >95) or severe AA (SALT core between 50-95), or moderate AA (SALT core between 25-50) or mild AA (SALT score <25) (Solomon, 2015).

The term “effective amount” as used herein means a therapeutically effective amount of one or more compositions, pharmaceutical compositions, therapeutic agents, active ingredients, compounds, etc of the disclosure, high enough to significantly positively modify the symptoms and/or condition to be treated, but low enough to avoid serious side effects (at a reasonable risk/benefit ratio), within the scope of sound medical judgment.

In the context of the present invention, the first pharmaceutical composition comprises a therapeutically effective amount of at least one inhibitor of a protein tyrosine kinase (PTK) involved in cytokine signalling. Preferably, the inhibitor of a protein tyrosine kinase (PTK) involved in cytokine signalling is a Janus kinase (JAK) inhibitor.

Over the past few years, various JAK inhibitors have been reported to have promising efficacy in various autoimmune disorders, including AA (Bose P. et al., 2017). Overexpression of JAK3 and, to a lesser extent, JAK1 and JAK2 was observed in skin biopsy specimens of patients with AA (Alves de Medeiros A K et al., 2016).

Janus kinase (JAK)-inhibitors is a new class of immunosuppressant drugs. The first JAK inhibitor, ruxolitinib, was approved in 2011 by the US Food and drug Administration (FDA) for the treatment of rheumatoid arthritis. Since then, several other JAK inhibitors have been developed and approved for the treatment of other autoimmune diseases.

Two JAK-inhibitors have been recently approved for treatment of AA, which is an autoimmune disease. Baricitinib was approved for the treatment of severe AA in adults, and ritlecitinib for the treatment of severe AA in adults and adolescents 12 years and older.

Severe AA defines as patients with Severity of Alopecia Tool (SALT) score ≥50. Patients with SALT score <50 are therefore not eligible to use JAK inhibitors. Patients who have a SALT <50 at start of treatment should be prescribed a drug which is authorized for patients with SALT <50. For patients with SALT ≥50 at start of treatment and prescribed an oral JAK inhibitor, an issue arises when their alopecia extent reaches SALT <50 since both drugs approved are allowed only for patients with severe AA (i.e., SALT ≥50).

It has been observed however that JAK inhibitors' discontinuation leads to rapid and significant disease relapse (Yan, 2022). Since JAK inhibitors' discontinuation leads to disease relapse, a continuation treatment which is allowed to be used in patients with SALT <50 is needed.

In the Phase 2/3 of the clinical trial conducted by the Inventors (RAAINBOW trial), the composition (herein “the second pharmaceutical composition”, preferably a topical formulation of the composition as described herein) comprising, as active ingredients, therapeutically effective amounts of an extract of Allium species, an extract of Citrus species, an extract of Paullinia species and an extract of Theobroma species was authorized to be evaluated not only in patients with severe AA, but also in patients with moderate AA (SALT 25-50). The composition could therefore be used as a continuation treatment after JAK inhibitors discontinuation.

Based on strong evidence, the Inventor's believe that using the composition after JAK inhibitors' discontinuation stands also on the fact that both drugs have complementary mechanisms of action.

The therapeutic efficacy of JAK inhibitors is based on their broad, relatively non-specific anti-inflammatory activities as a result of their ability to block or disrupt several signaling pathways used by type I and type II cytokines (Gilhar et al., 2019). As AA is a T-cell mediated autoimmune disorder, disruption of downstream signaling initiated by these pro-inflammatory cytokines, such as IFNγ, can hinder leucocyte recruitment to the hair follicle and block the release of cytotoxic granzymes which are responsible for hair loss (Stefanis et al., 2023) However, some of the T cells which invade the hair follicle remain there during treatment, as ‘resident memory T cells’ (Passeron et al., 2023) As a consequence, once treatment with JAK inhibitors is stopped, patients experience recurrence of alopecia. This relapse is mainly due to the “release of inhibition” of proinflammatory cytokines and other signaling molecules upon discontinuation of JAK inhibitors' treatment, causing excessive cell apoptosis, leading to premature termination of anagen, forcing the hair follicle (HF) entering catagen phase, the main event underlying AA and a critical indication of immune privilege collapse in AA development (Paus et al., 2018)

The second pharmaceutical composition of the invention not only has anti-inflammatory function, but also exerts its beneficial effects on hair follicle/perifollicular areas by normalizing the apoptotic process via upregulating cell expression of the anti-apoptotic protein Bcl-2 in the scalp towards the level identified in healthy subjects, thus prolonging anagen phase and preventing the premature onset of catagen. Meanwhile, the observed improved expression of Ki-67 in hair follicle cells after use of the composition indicates enhanced cell survival capacity, signifying higher number of active cycling cells (Cucé et al., 2011).

In one aspect, the JAK inhibitor is selected from the group comprising a JAK 1 inhibitor, a JAK 2 inhibitor, a JAK 3 inhibitor, and a tyrosine kinase 2 (TYK2) inhibitor, or a combination of one or more thereof (such as e.g. combination of JAK1/JAK2 inhibitors, JAK1/JAK3 inhibitors, and/or JAK2/JAK3 inhibitors).

Non limiting examples of combined JAK1/JAK2 inhibitors are selected from the group comprising baricitinib, ruxolitinib and deuruxolitinib.

Non limiting examples of JAK1 inhibitors are selected from the group comprising abrocitinib and upadacitinib.

Non limiting examples of JAK3 inhibitors are selected from the group comprising ritlecitinib and tofacitinib.

The first pharmaceutical composition is administered in a route selected from the group comprising intra-muscular injection, intra-peritoneal injection, an infusion, intravenous, intradermal, subcutaneous, oral, inhalation, transdermal, topical, transmucosal, nasal and rectal, or a combination thereof. In one aspect, the first pharmaceutical composition is administered by oral or topical route.

Suitable topical administration formulations include liquid or semi-liquid preparations suitable for penetration through the skin such as solutions, lotions, shake lotions, creams, ointments, gels, foams, transdermal patches, powders, solids, sponges, tapes, vapors, pastes, tinctures, microparticles, microcapsules, nanoparticles, liposomes, or emulsions.

In the context of the present invention, the second pharmaceutical composition of the invention comprises, as active ingredients, effective amounts of an extract of Allium species, an extract of Citrus species, an extract of Paullinia species and an extract of Theobroma species active Allium species, an extract of Citrus species, an extract of Paullinia species and an extract of Theobroma species.

The term extract, or aqueous-alcoholic extract, of Allium species refers particularly to aqueous-alcoholic extracts and native extracts (e.g. aqueous extracts) obtained from all species of the genus Allium (family Liliaceae) and especially Allium cepa.

The term extract, or aqueous-alcoholic extract, of Citrus species refers particularly to aqueous-alcoholic extracts and native extracts (e.g. aqueous extracts) obtained from all species of the genus Citrus (family Rutaceae) and especially Citrus lemon.

The term extract (atomised or not), or aqueous-alcoholic extract, of Paullinia species refers particularly to aqueous-alcoholic extracts and native extracts (e.g. aqueous extracts) obtained from all species of the genus Paullinia (family Sapindaceae) and especially Paullinia cupana.

The term extract (atomised or not), or aqueous-alcoholic extract, of Theobroma species refers particularly to aqueous-alcoholic extracts and native extracts (e.g. aqueous extracts) obtained from all species of the genus Theobroma (family Malvaceae) and especially Theobroma cacao.

In an aspect of the invention, the second pharmaceutical composition, or pharmaceutical composition for use, of the invention is administered topically, usually on external skin surface of the skull.

In a related aspect, the second pharmaceutical composition is usually formulated for topical administration. Preferably, the second pharmaceutical composition is mixed with diluents and/or excipients to formulate a composition for topical administration containing about 20% or more by weigh, based on the total weight of the second pharmaceutical composition.

Usually, the second pharmaceutical composition of the invention is administered topically, preferably on external skin surface of the skull, at least once per day, at least twice per day, or more.

Preferably, a volume comprised between about 0.5 ml and 2.5 ml of the second pharmaceutical composition formulated for topical administration is applied, at least once per day, at least twice per day, or more, in order to cover the whole scalp of the subject.

In one aspect, the second pharmaceutical composition, or second pharmaceutical composition for use, of the invention comprises from about 65% to about 93% by weight of an aqueous-alcoholic extract of Allium species; from about 5% to about 33% by weight of an aqueous-alcoholic extract of Citrus species; from about 0.25% to about 2.5% by weight of an aqueous extract of Paullinia species; and from about 0.25% to about 2.5% by weight of an aqueous-alcoholic extract of Theobroma species.

In a preferred aspect, the second pharmaceutical composition, or second pharmaceutical composition for use, of the invention comprises from about 65% to about 93% by weight of an aqueous-alcoholic extract of Allium cepa; from about 5% to about 33% by weight of an aqueous-alcoholic extract of Citrus lemon; from about 0.25% to about 2.5% by weight of an aqueous extract of Paullinia cupana; and from about 0.25% to about 2.5% by weight of an aqueous-alcoholic extract of Theobroma cacao.

In an even more preferred aspect, the second pharmaceutical composition, or second pharmaceutical composition for use, of the invention comprises about 87% by weight of an aqueous-alcoholic extract of Allium cepa; about 12% by weight of an aqueous-alcoholic extract of Citrus lemon; about 0.67% by weight of an aqueous extract of Paullinia cupana; and about 0.67% by weight of an aqueous-alcoholic extract of Theobroma cacao.

In one aspect, the second pharmaceutical composition of the invention, or second pharmaceutical composition for use, further contains excipients and/or diluents. Usually, excipients from about 0.05% to about 8.0% by weight of sodium chloride and from about 1% to about 40% by weight of glycerine are present, based on the total weight of the composition, preferably of the second pharmaceutical composition formulated for topical administration (i.e. topical pharmaceutical composition).

In one aspect, the second pharmaceutical composition of the invention, or second pharmaceutical composition for use, comprises from about 0.05% to about 8.0%, preferably from about 0.1% to about 7.0%, more preferably from about 0.4% to about 6.0%, and even more preferably from about 0.9% to about 3% by weight of sodium chloride, based on the total weight of the composition, preferably of the second pharmaceutical composition formulated for topical administration (i.e. topical pharmaceutical composition).

In one aspect, the second pharmaceutical composition of the invention, or second pharmaceutical composition for use, comprises from about 1% to about 20%, preferably from about 1.2% to about 15%, more preferably from about 1.8% to about 10%, and even more preferably from about 2% to about 5% by weight of glycerine, based on the total weight of the composition, preferably of the second pharmaceutical composition formulated for topical administration (i.e. topical pharmaceutical composition).

The second pharmaceutical composition of the invention suitable or formulated for topical administration includes liquid or semi-liquid preparations suitable for penetration through the skin such as solutions, lotions, shake lotions, creams, ointments, gels, foams, transdermal patches, powders, solids, sponges, tapes, vapors, pastes, tinctures, microparticles, microcapsules, nanoparticles, liposomes, or emulsions. Preferably, the compositions of the invention suitable or formulated for topical administration are in the form of solutions or lotions.

In some aspects, the one or more therapeutic effects surprisingly persist and even improve. As noted by the Inventors, the one or more therapeutically effects persist for at least 8 weeks, at least 12 weeks, at least 16 weeks, at least 20 weeks, at least 24 weeks or more after the last administration of the second pharmaceutical composition, as evidenced by measuring the severity of alopecia tool (SALT) score.

### Salt>95

In a preferred aspect, for patients whose hair loss extent is determined as being superior to SALT 95, the first pharmaceutical composition is administered and, only once hair loss extent has reached SALT 95, the second pharmaceutical composition is administered. The administration of the first pharmaceutical composition is then stopped after 24 to 36 weeks, preferably after 28 weeks, more preferably after 32 weeks, even more preferably after 36 weeks, and administration with the second pharmaceutical composition is maintained.

### Salt<95

In a preferred aspect, for patients whose hair loss extent is determined as being inferior to SALT 95, the first and second pharmaceutical compositions are co-administered. The administration of the first pharmaceutical composition is then stopped after 24 to 36 weeks, preferably after 28 weeks, more preferably after 32 weeks, even more preferably after 36 weeks, and treatment with the second pharmaceutical composition is maintained.

The present invention also contemplates a pharmaceutical composition for use in the treatment and/or prevention of hair loss comprising, as active ingredients, therapeutically effective amounts of an extract of Allium species, an extract of Citrus species, an extract of Paullinia species and an extract of Theobroma species, wherein said pharmaceutical composition is administered concomitantly with a pharmaceutical composition comprising a therapeutically effective amount of at least one Janus kinase (JAK) inhibitor to a subject in need thereof as described herein.

The present invention also contemplates a pharmaceutical composition comprising, as active ingredients, therapeutically effective amounts of an extract of Allium species, an extract of Citrus species, an extract of Paullinia species and an extract of Theobroma species as described herein for use in the treatment and/or prevention of hair loss in a subject in need thereof,
wherein said pharmaceutical composition is administered once the administration of a first pharmaceutical composition comprising a therapeutically effective amount of at least one Janus kinase (JAK) inhibitor is discontinued.

The present invention further contemplates methods of treatment and/or prevention of hair loss in a subject.

In one aspect, the method of treatment and/or prevention of hair loss in a subject comprises, administering i) a first pharmaceutical composition comprising a therapeutically effective amount of at least one Janus kinase (JAK) inhibitor and, ii) a second pharmaceutical composition comprising, as active ingredients, therapeutically effective amounts of an extract of Allium species, an extract of Citrus species, an extract of Paullinia species and an extract of Theobroma species,
wherein the first and second pharmaceutical compositions are administered concomitantly or staggered in time to a subject in need thereof as described herein.

In one aspect, the method of treatment and/or prevention of hair loss in a subject comprises, administering a second pharmaceutical composition comprising, as active ingredients, therapeutically effective amounts of an extract of Allium species, an extract of Citrus species, an extract of Paullinia species and an extract of Theobroma species, once the administration of a first pharmaceutical composition comprising a therapeutically effective amount of at least one Janus kinase (JAK) inhibitor is discontinued.

The present invention further contemplates the use of the pharmaceutical compositions of the invention in the manufacture of a medicament for the treatment and/or prevention of hair loss in a subject, comprising, administering i) a first pharmaceutical composition comprising a therapeutically effective amount of at least one Janus kinase (JAK) inhibitor and, ii) a second pharmaceutical composition comprising, as active ingredients, therapeutically effective amounts of an extract of Allium species, an extract of Citrus species, an extract of Paullinia species and an extract of Theobroma species, wherein the first and second pharmaceutical compositions are administered concomitantly or staggered in time to a subject in need thereof as described herein.

The present invention also contemplates the use of a pharmaceutical composition comprising, as active ingredients, therapeutically effective amounts of an extract of Allium species, an extract of Citrus species, an extract of Paullinia species and an extract of Theobroma species, of the invention, in the manufacture of a medicament for the treatment and/or prevention of hair loss in a subject, wherein said pharmaceutical composition is administered once the administration of a first pharmaceutical composition comprising a therapeutically effective amount of at least one Janus kinase (JAK) inhibitor is discontinued.

The invention also contemplates kits for the treatment and/or prevention of hair loss as described herein. In one aspect of the invention, the kit comprises the pharmaceutical compositions, or compositions for use, of the invention.

The kits of the invention may also comprise a container and a label or package insert on or associated with the container. Suitable containers include, for example, bottles, vials, syringes, dispensers, a spray applicator, etc. The containers may be formed from a variety of materials such as glass or plastic.

In one aspect, the kit comprises at least two containers, one for each composition of the invention.

The label or package insert may comprise instructions for use thereof. Instructions included may be affixed to packaging material or may be included as a package insert. While the instructions are typically written or printed materials they are not limited to such. Any medium capable of storing such instructions and communicating them to an end user is contemplated by this disclosure.

The present disclosure is to be considered as in all aspects illustrated and not restrictive, the scope of the invention being indicated by the appended Claims, and all changes which come within the meaning and range of equivalency are intended to be embraced therein.

### EXAMPLES

### Material and Methods

### Composition

The composition comprises about 87% by weight of an aqueous-alcoholic extract of Allium cepa; about 12% by weight of an aqueous-alcoholic extract of Citrus limon; about 0.67% by weight of an aqueous extract of Paullinia cupana; and about 0.67% by weight of an aqueous-alcoholic extract of Theobroma cacao.

### Topical Formulation

The final topical solution product contains at least 20% of the composition as described above. The remainder of the finished product consists of water and regular excipients found in other cutaneous solutions, such as betaine, glycerin, sodium chloride, etc. . . .

A finished cutaneous solution product (LH-8) containing 22.25% of the composition comprising, as active ingredients, effective amounts of an extract of Allium species, an extract of Citrus species, an extract of Paullinia species and an extract of Theobroma species has been evaluated in a clinical trial evaluating its safety and efficacy in subjects presenting AA (RAAINBOW study, ClinicalTrials.gov identifier NCT03240627). Approximately 1 mL of the composition was applied to whole scalp twice per day, at approximately 12-hour intervals (e.g. in the morning and in the evening)

The RAAINBOW study was a double-blind, randomized, multi-center study versus placebo. 107 subjects (males and females) were enrolled and serve for safety analysis. Among them 62 within inclusion criteria (i.e. moderate to severe AA) representing the Full Analysis Set population (FAS) were analyzed for efficacy.

### Example 1 Results

Below is the summary of the safety analysis.

| | Composition (N = 71) | | Placebo (N = 36) | |
|---|---|---|---|---|
| Characteristics | n % | E | n % | E |
| Any AEs (Adverse Events) | 28 (39.4%) | 62 | 17 (47.2%) | 42 |
| Any SAEs | 0 | | 1 (2.8%) | 2 |
| Any TEAEs | 28 (39.4%) | 58 | 17 (47.2%) | 38 |
| Drug-related TEAEs | 4 (5.6%) | 5 | 4 (11.1%) | 4 |
| Severe TEAEs | 1 (1.4%) | 1 | 1 (2.8%) | 2 |
| Serious TEAEs | 0 | | 1 (2.8%) | 2 |
| TEAEs Leading to Drug Withdrawn | 1 (1.4%) | 1 | 0 | |
| TEAEs Leading to Drug Interruption | 1 (1.4%) | 1 | 0 | |
| TEAEs Leading to Death | 0 | | 0 | |

[1] Percentages are computed using N provided in the Column header.
[2] AE: Adverse Event, TEAE: Treatment Emergent Adverse Event, n: Number of subjects; E- Number of Events

No subjects of the composition group presented a serious adverse event (AE). Only 5,6% of composition-treated subjects presented AEs presumably related to the drug (VS 11,1% in the Placebo group). One AE was severe, consisting of severe scalp and face eczema. However, eczema is the most common comorbidity of AA and patients with AA are more likely to present atopic dermatitis, eczema (17.4% vs. 2.2% controls) (Conic, 2020). All other AEs were mild, moderate, local and transient. None of side-effects observed with JAK inhibitors were reported.

No subjects of the composition group presented a serious adverse event (AE). Only 5.6% of compositing-treated subjects presented an AEs presumably related to the drug (VS 11.1% in the Placebo group). One AE was severe, consisting of severe scalp and face eczema. However, eczema is the most common comorbidity of AA and patients with AA are more likely to have atopic dermatitis, eczema (17.4% vs. 2.2% controls) (Conic, 2020). All other AEs were mild, moderate, local and transient. None of side-effects observed with JAK inhibitors have been reported.

The composition is perfectly safe in the treatment of AA and warrants therefore no treatment discontinuation periods. Therefore, the composition can be used in chronic fashion with no risk to users' health.

Since the composition has demonstrated clinical efficacy in the treatment of patients with AA with a hair loss extent under 95% (SALT <95), while presenting no health risks, the invention consists in switching treatment, from JAK inhibitor to the composition, in order to maintain patient's recovery from the disease, while ending his exposure to a drug which is documented to pose long term health risks. This novel regimen is plausible since the composition seemed to offer a comparable chance of disease recovery, as presented below.

### Janus Kinase (JAK) Inhibitor Presentation

JAK inhibitor have been evaluated for the treatment of AA patients presenting hair loss extent of 50% and above (SALT>50). In clinical trials, several JAK inhibitor were evaluated after 24 to 36 weeks of treatment and demonstrated clinical efficacy against placebo.

AA was evaluated through the scalp alopecia areata severity score, known as the SALT (Severity of Alopecia Tool) score, based on global standardized scalp photographs. SALT score was developed in 2004 by Olsen (Olsen, 2004) “to help facilitate well-controlled clinical trials for alopecia areata”. It consists of a standardized method to assess the extent of alopecia, a SALT score of 100% consisting of full-baldness. It is based on a 4 pictures-based scoring system, where each side of the head is split into 4 quadrants, and scored by the investigator accordingly. The SALT score is the standard measurement endpoint, used by all trials evaluating new treatments efficacy in AA (see FIG. 1)

So far, three JAK inhibitors have been evaluated in late-stage trials in patients with SALT>50 AA: baricitinib, ritlecitinib, deuruxolitinib. For the three JAK inhibitors, the proportion of patients reaching SALT<20, or hair loss extent of less than 20%, was measured, and consisted of the study primary outcome.

For baricitinib, the estimated percentage of patients with a SALT score of 20 or less at week 36 was 38.8% with 4-mg baricitinib, 22.8% with 2-mg baricitinib, and 6.2% with placebo in a first trial, and 35.9%, 19.4%, and 3.3%, respectively, in a second trial (King 2021).

For ritlecitinib, at week 24, 31% of patients in the ritlecitinib 200 mg+50 mg group, 22% of patients in the 200 mg+30 mg group, 23% patients in the 50 mg group, 14% of patients in the 30 mg group, and 2% of patients in the placebo group had a response based on SALT score 20 or less (King, 2023).

For deuruxolitinib, the proportion of patients achieving a SALT score of 20 or less was 38.3% in the 12 mg twice-daily dose group and 33.0% in the 8 mg twice-daily dose group, compared to 0.8% of patients in the placebo group, at the 24-week endpoint (press release).

For the composition, the proportion of patients with a SALT score of 20 or less was 21.2% in the composition group and 5.3% in the placebo group at week 24, and 46.7% and 9.1% respectively at week 48. While patients in the JAK inhibitors trial had an AA extent above 50%, patients treated with the composition had an extent of hair loss of 25-95% (SALT 25-95).

Since JAK inhibitor discontinuation triggers disease relapse within 3 months (Yann, 2022), the protocol for interrupting JAK inhibitor and switching treatment to the composition is defined as to minimize disease relapse.

### Example 2

### Combination Treatment—Hair Loss Extent Superior to SALT 95

For AA patients whose hair loss extent is superior to SALT 95, treatment regimen consists in starting with the approved JAK inhibitor baricitinib at 4 mg once a day, and the second pharmaceutical composition is administered once hair loss extent has reached SALT 95, at 1 ml, twice a day. The JAK inhibitor treatment is then interrupted after 36 weeks, and treatment with the second pharmaceutical composition is maintained.

### Example 3

### Combination Treatment—Hair Loss Extent Inferior to SALT 95

For AA patients whose hair loss extent is inferior to SALT 95, treatment regimen consists in administering, simultaneously or concomitantly at 1 ml, twice a day. The JAK inhibitor treatment is then interrupted after 36 weeks, and treatment with the second pharmaceutical composition is maintained.

### Results

In both examples, hair regrowth continued overtime, with no long-term health risk from the long term use of the compositions of the invention.

### REFERENCES

https://pi.lilly.com/us/olumiant-uspi.pdf
Alves de Medeiros A K, Speeckaert R, Desmet E, van Gele M, de Schepper S, Lambert J. JAK3 as an emerging target for topical treatment of inflammatory skin diseases. PLOS One. 2016; 11 (10): e0164080.
Bose P, Verstovsek S. JAK2 inhibitors for myeloproliferative neoplasms: what is next? Blood. 2017; 130 (2): 115-125.
Conic R Z, Tamashunas N L, Damiani G, Fabbrocini G, Cantelli M; Young Dermatologists Italian Network; Bergfeld W F. Comorbidities in pediatric alopecia areata. J Eur Acad Dermatol Venereol. 2020; 34 (12): 2898-2901.
Cucé LC, Rodrigues C J, Patriota R C R. Cellium® GC: evaluation of a new natural active ingredient in 210 mg/ml topical solution, through scalp biopsy. Surgical Cosmetic Dermatology 2011; 3:123-128.
Gilhar A, Keren A, Paus R. JAK inhibitors and alopecia areata. Lancet. 2019 Jan. 26; 393 (10169): 318-319. doi: 10.1016/S0140-6736 (18) 32987-8.
Haughton R D, Herbert S M, Ji-Xu A, Downing L, Raychaudhuri S P, Maverakis E. Janus kinase inhibitors for alopecia areata: A narrative review. Indian J Dermatol Venereol Leprol. 2023 November-December; 89 (6): 799-806.
Hoisnard L, Lebrun-Vignes B, Maury S, Mahevas M, El Karoui K, Roy L, Zarour A, Michel M, Cohen J L, Amiot A, Claudepierre P, Wolkenstein P, Grimbert P, Sbidian E. Adverse events associated with JAK inhibitors in 126,815 reports from the WHO pharmacovigilance database. Sci Rep. 2022 May 3; 12 (1): 7140.
King B, Ko J, Forman S, Ohyama M, Mesinkovska N, Yu G, McCollam J, Gamalo M, Janes J, Edson-Heredia E, Holzwarth K, Dutronc Y. Efficacy and safety of the oral Janus kinase inhibitor baricitinib in the treatment of adults with alopecia areata: Phase 2 results from a randomized controlled study. J Am Acad Dermatol. 2021 October; 85 (4): 847-853.
King B, Zhang X, Harcha W G, Szepietowski J C, Shapiro J, Lynde C, Mesinkovska N A, Zwillich S H, Napatalung L, Wajsbrot D, Fayyad R, Freyman A, Mitra D, Purohit V, Sinclair R, Wolk R. Efficacy and safety of ritlecitinib in adults and adolescents with alopecia areata: a randomised, double-blind, multicentre, phase 2b-3 trial. Lancet. 2023 Apr. 13: S0140-6736 (23) 00222-2.
Olsen E A, Hordinsky M K, Price V H, Roberts J L, Shapiro J, Canfield D, Duvic M, King L E Jr, McMichael A J, Randall V A, Turner M L, Sperling L, Whiting D A, Norris D; National Alopecia Areata Foundation. Alopecia areata investigational assessment guidelines—Part II. National Alopecia Areata Foundation. J Am Acad Dermatol. 2004 September; 51 (3): 440-7.
Passeron T, King B, Seneschal J, Steinhoff M, Jabbari A, Ohyama M, Tobin D J, Randhawa S, Winkler A, Telliez J B, Martin D, Lejeune A. Inhibition of T-cell activity in alopecia areata: recent developments and new directions. Front Immunol. 2023 Nov. 6; 14:1243556.
Paus R, Bulfone-Paus S, Bertolini M. Hair Follicle Immune Privilege Revisited: The Key to Alopecia Areata Management. J Investig Dermatol Symp Proc. 2018 January; 19 (1): S12-S17. doi: 10.1016/j.jisp.2017.10.014.
Solomon J A. Development of Uniform Protocol for Alopecia Areata Clinical Trials. J Investig Dermatol Symp Proc. 2015 November; 17 (2): 63-6.
Yan D, Fan H, Chen M, Xia L, Wang S, Dong W, Wang Q, Niu S, Rao H, Chen L, Nie X, Fang Y. The efficacy and safety of JAK inhibitors for alopecia areata: A systematic review and meta-analysis of prospective studies. Front Pharmacol. 2022 Aug. 24; 13:950450.
https://www.fda.gov/drugs/drug-safety-and-availability/fda-requires-warnings-about-increased-risk-serious-heart-related-events-cancer-blood-clots-and-death
https://www.ema.europa.eu/en/news/ema-confirms-measures-minimise-risk-serious-side-effects-janus-kinase-inhibitors-chronic
https://ir.concertpharma.com/news-releases/news-release-details/late-breaking-phase-3-data-aad-2023-show-oral-investigational

1. A method for the treatment and/or prevention of hair loss, the method comprising administering to a subject in need thereof:
i) a first pharmaceutical composition comprising a therapeutically effective amount of at least one Janus kinase (JAK) inhibitor and,
ii) a second pharmaceutical composition comprising, as active ingredients, therapeutically effective amounts of an extract of Allium species, an extract of Citrus species, an extract of Paullinia species and an extract of Theobroma species,
wherein the first and second pharmaceutical compositions are administered concomitantly or staggered in time to the subject in need thereof.

2. The method according to claim 1, wherein the first pharmaceutical composition is administered for a period of time necessary to detect one or more therapeutic effects comprising slowing down the loss of hair, stimulating its growth and/or increasing the density of hair, as evidenced by measuring the severity of alopecia tool (SALT) score.

3. The method according to claim 2, wherein the period of time necessary to detect one or more therapeutically effects is comprised between about 16 to about 48 weeks.

4. The method according to claim 1, wherein the administration of the first composition is discontinued after 24 to 36 weeks.

5. The method according to claim 1, wherein the first and second compositions are coadministered concomitantly for at least 1 hour, at least 1 day, at least 4 days, at least one week, at least 2 weeks, at least 3 weeks, at least 5 weeks, at least 10 weeks, at least 15 weeks, at least 20 weeks, at least 25 weeks, at least 36 weeks or more.

6. The method according to claim 1, wherein the first and second compositions are administered staggered in time and wherein the second pharmaceutical composition is administered after the last administration of the first pharmaceutical composition.

7. The method according to claim 6, wherein the second pharmaceutical composition is administered about one hour, about 1 day, about 4 days, about one week, about 2 weeks, about 3 weeks, about 5 weeks, about 10 weeks, about 15 weeks, about 20 weeks or more, after the last administration of the first pharmaceutical composition.

8. The method according to claim 1, wherein the hair loss is an immune-mediated disease.

9. The method according to claim 8, wherein the immune-mediated disease is an autoimmune disease.

10. The method according to claim 9, wherein the autoimmune disease is alopecia aerata.

11. (canceled)

12. The method according to claim 2, wherein a decrease of at least about 2% or more, at least about 5% or more, at least about 10% or more, at least about 15% or more, at least about 20% or more, at least about 30% or more, at least about 40% or more, or at least about 50% or more in SALT score is detected when compared to the subject's baseline SALT score determined before starting the administration of the first pharmaceutical composition.

13. The method according to claim 1, wherein the JAK inhibitor comprises a JAK 1 inhibitor, a JAK 2 inhibitor, and a JAK 3 inhibitor, or a combination of one or more thereof.

14. The method according to claim 13, wherein the JAK1 inhibitor comprises abrocitinib, upadacitinib, or a combination thereof.

15. The method according to claim 13, wherein the JAK3 inhibitor comprises ritlecitinib, tofacitinib, or a combination thereof.

16. The method according to claim 13, wherein the combined JAK1/JAK2 inhibitors comprise baricitinib, ruxolitinib, deuruxolitinib, or a combination of two or more thereof.

17. The method according to claim 1, wherein the second pharmaceutical composition comprises from about 65% to about 93% by weight of an aqueous-alcoholic extract of Allium species; from about 5% to about 33% by weight of an aqueous-alcoholic extract of Citrus species; from about 0.25% to about 2.5% by weight of an aqueous extract of Paullinia species; and from about 0.25% to about 2.5% by weight of an aqueous-alcoholic extract of Theobroma species.

18. The method according to claim 1, wherein the first pharmaceutical composition is administered in a route comprising intra-muscular injection, intra-peritoneal injection, an infusion, intravenous, intradermal, subcutaneous, oral, inhalation, transdermal, topical, transmucosal, nasal, rectal, or a combination thereof.

19. The method according to claim 1, wherein the second pharmaceutical composition is administered topically.

20. The method according to claim 1, wherein the second pharmaceutical composition is mixed with diluents and/or excipients to formulate a composition for topical administration containing about 20% or more by weight, based on the total weight of the topical pharmaceutical composition.

21. The method according to claim 1, wherein the second pharmaceutical composition comprises from about 0.05% to about 8.0% by weight of sodium chloride, based on the total weight of the topical pharmaceutical composition.

22. The method according to claim 1, wherein the second pharmaceutical composition comprises from about 1% to about 20% by weight of glycerine, based on the total weight of the topical pharmaceutical composition.

23. The method according to claim 1, wherein if the hair loss extent is determined as being superior to SALT 95,
i) the first pharmaceutical composition is administered and,
ii) once hair loss extent has reached SALT 95, the second pharmaceutical composition is administered.

24. The method according to claim 23, wherein the administration of the first pharmaceutical composition is discontinued after 24 to 36 weeks, and the administration of the second pharmaceutical composition is maintained.

25. The method according to claim 1, wherein if the hair loss extent is determined as being inferior to SALT 95, the first and second pharmaceutical compositions are co-administered.

26. The method according to claim 25, wherein the administration of the first pharmaceutical composition is discontinued after 24 to 36 weeks, and the administration of the second pharmaceutical composition is maintained.

27.-31. (canceled)

32. A kit for the treatment and/or prevention of hair loss comprising:
i) a first pharmaceutical composition comprising a therapeutically effective amount of at least one Janus kinase (JAK) inhibitor, and
ii) a second pharmaceutical composition comprising, as active ingredients, therapeutically effective amounts of an extract of Allium species, an extract of Citrus species, an extract of Paullinia species and an extract of Theobroma species.

33. The kit of claim 32, further comprising one or more containers, label, or package inserts on, or associated with, the one or more containers.

34.-36. (canceled)
